(19) United States
(12) Patent Application Publication
(10) Pub. No.: US 2014/0024905 A1
(43) Pub. Date: Jan. 23, 2014

(54) APPARATUS, SYSTEMS, AND METHODS FOR TISSUE OXIMETRY AND PERFUSION IMAGING

(71) Applicant: THE REGENTS OF THE UNIVERSITY OF CALIFORNIA, Oakland, CA (US)

(72) Inventors: Majid Sarrafzadeh, Anaheim Hills, CA (US); William Kaiser, Los Angeles, CA (US); Barbara Bates-Jensen, Pasadena, CA (US); Alireza Mehrnia, Los Angeles, CA (US); Bijan Mapar, Reston, VA (US); Frank Wang, Cupertino, CA (US)

(73) Assignee: THE REGENTS OF THE UNIVERSITY OF CALIFORNIA, Oakland, CA (US)

(21) Appl. No.: 13/942,649

(22) Filed: Jul. 15, 2013

Related U.S. Application Data
(63) Continuation of application No. PCT/US2012/021919, filed on Jan. 19, 2012.
(60) Provisional application No. 61/434,014, filed on Jan. 19, 2011.

(51) Int. Cl. A61B 5/1455; A61B 5/00

(52) U.S. Cl. CPC A61B 5/14552; A61B 5/742; A61B 5/7203
USPC 600/328

(57) ABSTRACT

A compact perfusion scanner and method of characterizing tissue health status are disclosed that incorporate pressure sensing components in conjunction with the optical sensors to monitor the level of applied pressure on target tissue for precise skin/tissue blood perfusion measurements and oximetry. The systems and methods allow perfusion imaging and perfusion mapping (geometric and temporal), signal processing and pattern recognition, noise cancelling and data fusion of perfusion data, scanner position and pressure readings.

### CROSS-REFERENCE TO RELATED APPLICATIONS

This application is a 35 U.S.C. §111(a) continuation of PCT international application number PCT/US2012/021919 filed on Jan. 19, 2012, incorporated herein by reference in its entirety, which is a nonprovisional of U.S. provisional patent application Ser. No. 61/434,014 filed on Jan. 19, 2011, incorporated herein by reference in its entirety. Priority is claimed to each of the foregoing applications.

The above-referenced PCT international application was published as PCT International Publication No. WO 2012/100090 on Jul. 26, 2012 and republished on Sep. 13, 2012, and is incorporated herein by reference in its entirety.

### STATEMENT REGARDING FEDERALLY SPONSORED RESEARCH OR DEVELOPMENT

Not Applicable

### INCORPORATION-BY-REFERENCE OF MATERIAL SUBMITTED IN COMPUTER PROGRAM APPENDIX

Appendix A referenced herein is a computer program listing in a text file entitled “UC_{—}2011_{—}037_{—}2_US_source_code_listing.txt” created on Jun. 25, 2013 and having a 27 kb file size. The computer program code, which exceeds 300 lines, is submitted as a computer program listing appendix through EFS-Web and is incorporated herein by reference in its entirety.

### NOTICE OF MATERIAL SUBJECT TO COPYRIGHT PROTECTION

A portion of the material in this patent document is subject to copyright protection under the copyright laws of the United States and of other countries. The owner of the copyright rights has no objection to the facsimile reproduction by anyone of the patent document or the patent disclosure, as it appears in the United States Patent and Trademark Office publicly available file or records, but otherwise reserves all copyright rights whatsoever. The copyright owner does not hereby waive any of its rights to have this patent document maintained in secrecy, including without limitation its rights pursuant to 37 C.F.R. §1.14.

### BACKGROUND OF THE INVENTION

1. Field of the Invention

This invention pertains generally to tissue oximetry, and more particularly to tissue oximetry and perfusion imaging.

2. Description of Related Art

Patients' skin integrity has long been an issue of concern for nurses and in nursing homes. Maintenance of skin integrity has been identified by the American Nurses Association as an important indicator of quality nursing care. Meanwhile, ulcers, and specifically venous and pressure ulcers, remain major health problems, particularly for hospitalized older adults. Detecting early wound formation is an extremely challenging and expensive problem.

When age is considered along with other risk factors, the incidences of these ulcers are significantly increased. Overall incidence of pressure ulcers for hospitalized patients ranges from 2.7% to 29.5%, and rates of greater than 50% have been reported for patients in intensive care settings. In a multicenter cohort retrospective study of 1,803 older adults discharged from acute care hospitals with selected diagnoses, 13.2% (i.e., 164 patients) demonstrated an incidence of stage I ulcers. Of those 164 patients, 38 (16%) had ulcers that progressed to a more advanced stage.

Pressure ulcers additionally have been associated with an increased risk of death within one year after hospital discharge. The estimated cost of treating pressure ulcers ranges from $5,000 to $40,000 for each ulcer, depending on severity. Meanwhile, venous ulcers can also cause significant health problems for hospitalized patients, especially in older adults. As many as 3% of the population suffer from leg ulcers, while this figure rises to 20% in those over 80 years of age. The average cost of treating a venous ulcer is estimated at $10,000, and can easily rise as high as $20,000 without effective treatment and early diagnosis.

Once a patient has been afflicted by a venous ulcer, the likelihood of the wound recurring is also extremely high, and ranges from 54% to 78%. This means that venous ulcers can have severely negative effects on those who suffer from them, significantly reducing quality of life and requiring extensive treatment. The impact of venous ulcers is often underestimated, despite accounting for as much as 2.5% of the total health care budget.

The high cost and incidence rates of venous ulcers, coupled with the difficulty in treating them, mark an extremely good opportunity to introduce a low cost, non-invasive system capable of early detection. While traditional laser Doppler systems are able to deliver relatively accurate and reliable information, they cannot be used for continuous monitoring of patients, since they require bulky and extremely expensive equipment. Such solutions that are too expensive or difficult to deploy significantly limit adoption.

Hence, there is a need to develop a monitoring and preventive solution to scan the tissue and measure tissue perfusion status as a measure for the level of oxygen distribution and penetration throughout the tissue as an indicator of tissue health. Accordingly, an object of the present invention is the use of photoplethysmographic in conjunction with pressure sensor signals to monitor perfusion levels of patients suffering from or at risk of venous ulcers.

### BRIEF SUMMARY OF THE INVENTION

The systems and methods of the present invention include a compact perfusion scanner configured to scan and map tissue blood perfusion as a mean to detect and monitor the development of ulcers. The device incorporates a platform, a digital signal processing unit, a serial connection to a computer, pressure sensor, pressure metering system, an LED and photodiode sensor pair and a data explorer visual interface.

The systems and methods of the present invention provide effective preventive measures by enabling early detection of ulcer formation or inflammatory pressure that would otherwise have not been detected for an extended period, thus increasing risk of infection and higher stage ulcer development.

In a preferred embodiment, the compact perfusion scanner and method of characterizing tissue health status according to the present invention incorporates pressure sensing components in conjunction with the optical sensors to monitor the level of applied pressure on target tissue for precise skin/tissue blood perfusion measurements and oximetry. The systems and methods of the present invention enable new capabilities including but not limited to: measurement capabilities such as perfusion imaging and perfusion mapping (geometric and temporal), signal processing and pattern recognition, automatic assurance of usage via usage tracking and pressure imaging, as well as data fusion.

One particular benefit of the sensor-enhanced system of the present invention is the ability to better manage each individual patient, resulting in a timelier and more efficient practice in hospitals and even nursing homes. This is applicable to patients with a history of chronic wounds, diabetic foot ulcers, pressure ulcers or post-operative wounds.

In addition, alterations in signal content may be integrated with the activity level of the patient, the position of patient's body and standardized assessments of symptoms. By maintaining the data collected in these patients in a signal database, pattern classification, search, and pattern matching algorithms may be used to better map symptoms with alterations in skin characteristics and ulcer development.

An aspect is an apparatus for monitoring perfusion oxygenation of a target tissue region of a patient, comprising: a scanner comprising: a planar sensor array; the sensor array configured to be positioned in contact with a surface of the target tissue region; the sensor array comprising one or more LED's configured to emit light into the target tissue region at a wavelength keyed for hemoglobin; the sensor array comprising one or more photodiodes configured to detect light reflected from the LED's; and a data acquisition controller coupled to the one or more LED's and to the one or more photodiodes for controlling the emission and reception of light from the sensor array to obtain perfusion oxygenation data associated with the target tissue region.

Another aspect is a system for monitoring perfusion oxygenation of a target tissue region of a patient, comprising: (a) a scanner comprising: a planar sensor array; the sensor array configured to be positioned in contact with a surface of the target tissue region; the sensor array comprising one or more light sources configured to emit light into the target tissue region at a wavelength keyed for hemoglobin; the sensor array comprising one or more sensors configured to detect light reflected from the light sources; a pressure sensor coupled to the sensor array; the pressure sensor configured to obtain pressure readings of the sensor array's contact with a surface of the target tissue region; and (b) a data acquisition controller coupled to the one or more sensors and for controlling the emission and reception of light from the sensor array to obtain perfusion oxygenation data associated with the target tissue; and (c) a processing module coupled to the data acquisition controller; (d) the processing module configured to control sampling of the pressure sensor and sensor array for simultaneous acquisition of perfusion oxygenation data and pressure sensor data to ensure proper contact of the scanner with the surface of the target tissue region.

A further aspect is a method for performing real-time monitoring of perfusion oxygenation of a target tissue region of a patient, comprising: positioning a sensor array in contact with a surface of the target tissue region; emitting light from lights sources in the sensor array into the target tissue region at a wavelength keyed for hemoglobin; receiving light reflected from the light sources; obtaining pressure data associated with the sensor array's contact with a surface of the target tissue region; obtaining perfusion oxygenation data associated with the target tissue region; and sampling the perfusion oxygenation data and pressure data to ensure proper contact of the sensor array with the surface of the target tissue region.

It is appreciated that the systems and methods of the present invention are not limited to the specific condition of ulcer or wound, but may have broad application in all forms of wound management, such as skin diseases or treatments.

Further aspects of the invention will be brought out in the following portions of the specification, wherein the detailed description is for the purpose of fully disclosing preferred embodiments of the invention without placing limitations thereon.

### BRIEF DESCRIPTION OF THE SEVERAL VIEWS OF THE DRAWING(S)

The invention will be more fully understood by reference to the following drawings which are for illustrative purposes only:

FIG. 1 shows a preferred embodiment of a perfusion oxygenation monitoring (POM) system for analyzing a region of tissue in accordance with the present invention

FIGS. 2A and 2B illustrate front and right perspective views of the perfusion hardware printed circuit board of the present invention.

FIG. 3 illustrates an exemplary LED emitter in accordance with the present invention.

FIG. 4 illustrates LED driver circuit in accordance with the present invention.

FIG. 5 illustrates an exemplary photodiode read circuit configured for reading the signal from photodiode sensor array.

FIG. 6 illustrates a calibration setup for calibration of the pressure sensor.

FIG. 7 shows a plot of results from the pressure verification trials of weights of 50 g, 100 g, 200 g and 500 g on a single sensor.

FIG. 8 is a plot showing measured pressure response curve, interpolated curve (exponential), and the point where the pressure sensor is specified to saturate.

FIG. 9 shows results from pressure verification trials on a second 1-pound sensor.

FIG. 10 is a plot showing raw pressure response curves, and various fits.

FIG. 11 illustrates a PC setup for running the perfusion oxygenation monitoring (POM) system of the present invention.

FIG. 12 shows a screenshot of the hardware configuration module interface in accordance with the present invention.

FIG. 13 shows a screenshot of the graphical user interface in accordance with the present invention.

FIG. 14 shows an exemplary interpolation performed via a Kriging algorithm.

FIG. 15 shows a schematic diagram of a marker pattern used for testing the feature extraction module.

FIG. 16 illustrates the setup of FIG. 15 overlaid on an image.

FIG. 17 illustrates a block diagram of a method for outputting a mapped and interpolated perfusion image.

FIG. 18 shows an example of heterodyning used to help eliminate in-band noise in accordance with the present invention.

FIG. 19 is a plot of the theoretical response of the subtraction method of FIG. 18 in relation to noise and correction frequency.

FIG. 20 is a plot of the frequency response of the subtraction method shown on a dB scale.

FIG. 21 shows results from employing noise subtraction on a high frequency LED drive signal, and averaging several LED drive periods to obtain similar data rates as before.

FIG. 22 illustrates a zoomed view of FIG. 21.

FIG. 23 shows a sample of the time domain signals used for comparison of neck and thumb tissue measurements.

FIG. 24 shows the frequency domain representation of the measured signals.

FIG. 25 shows results from extracted plethysmograph signals of the forehead.

FIG. 26 shows a comparison of readings of extracted plethysmograph signals from under the knuckle on the thumb.

FIG. 27 shows results from varying pressure using the reflectance sensor on the neck.

FIG. 28 shows the results from both over and to the side of the black tape.

### DETAILED DESCRIPTION OF THE INVENTION

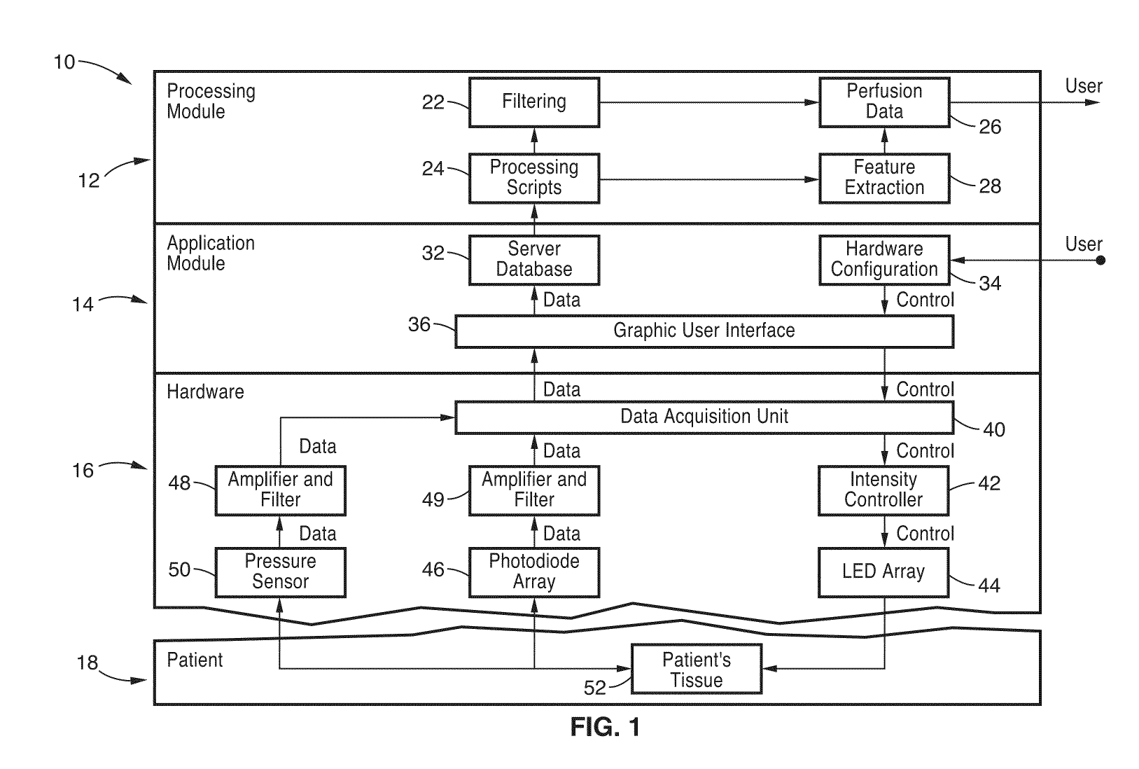

FIG. 1 shows a preferred embodiment of a perfusion oxygenation monitoring (POM) system 10 for analyzing a region of tissue 52 of a patient 18 in accordance with the present invention. System 10 generally comprises six primary components: red/infrared LED array 44, photodiode array 46, pressure sensor 50, pressure metering system 48 (which includes amplification and filtering circuitry), data acquisition unit 40, digital signal processing module 12 and application module 14 having a user interface.

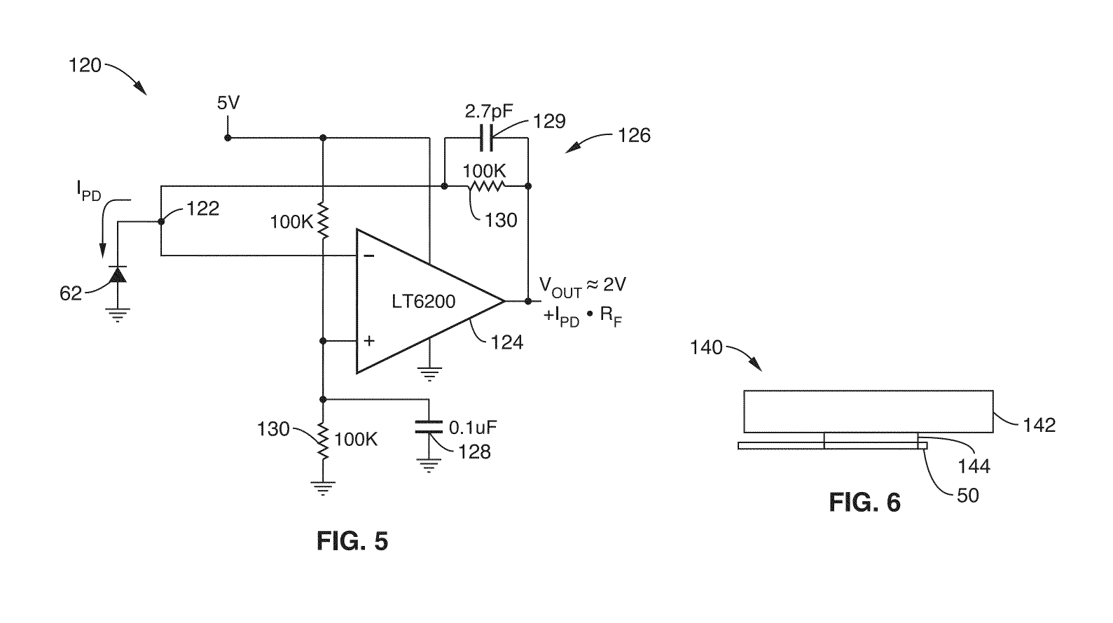

The system 10 comprises sensing hardware component 16 that includes arrays of emitters/sensors (44, 46, 50) and data acquisition unit 40, preferably in a handheld enclosure (not shown). The LED array 44 and photodiode arrays 46 coupled to the data acquisition unit 40 (e.g. through cabling or wireless connection) can be physically configured in a variety of arrays. The data acquisition unit 40 is preferably capable of interfacing with a large number of individual LEDs and photodiodes. Signal amplification and filtering unit 49 may be used to condition the photodiode signal/data prior to being received by the data acquisition unit 40. In a preferred embodiment, the photodiode signal amplification and filtering unit 49 may comprise a photodiode read circuit 120 shown in FIG. 5 and described in further detail below.

Sensing/scanning hardware component 16 may also include an intensity controller 42 for controlling the output of LED array 44. Intensity controller 42 preferably comprises LED driver circuit 100 shown in FIG. 4, and described in further detail below.

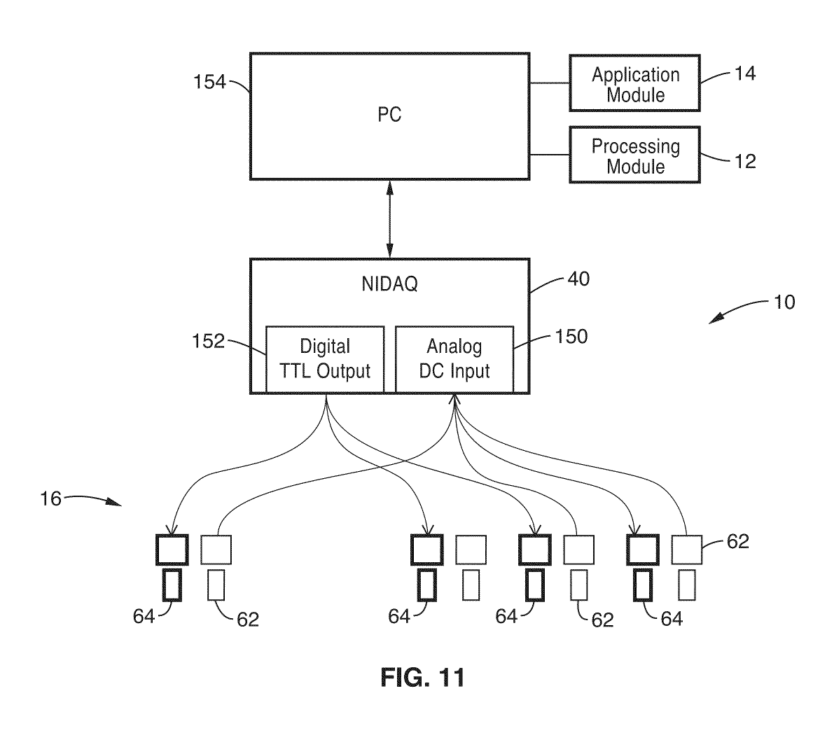

The data acquisition system 40 also interfaces with application module 14 on PC 154 (see FIG. 11), allowing a user to configure the LED array 44 signaling as well as sampling rate of the signal from photodiode array 46 via a hardware configuration module 34 that is viewed through the graphical user interface 36. Data acquired from DAC 40 is preferably stored in a database 32 for subsequent processing.

The pressure sensor 50 is configured to measure the pressure applied from the hardware package 16 on to the patient's tissue, such that pressure readings may be acquired to maintain consistent and appropriate pressure to the skin 52 while measurements are being taken. The pressure sensor 50 may be coupled to pre-conditioning or metering circuitry 48 that includes amplification and filtering circuitry to process the signal prior to being received by the data acquisition controller 40.

The LED arrays 44 are configured to project light at wavelengths keyed for hemoglobin in the target tissue 52, and the photodiode sensor arrays 46 measure the amount of light that passes through tissue 52.

The signal processing module 12 then further processes and filters the acquired data via processing scripts 24 and filtering module 22. The signal processing module 12 further comprises a feature extraction module 28, which may be output to visual interface 36 for further processing and visualization. A perfusion data module 26 converts data into a Plethysmograph waveform, which may be displayed on a monitor or the like (not shown). The interface 36 and processing module 12 may also be configured to output an overlay image of the tissue and captured perfusion data 26.

In order to produce the wavelengths of light corresponding to deoxy and oxyhemoglobin absorption, the system 12 preferably uses light emitting diodes for the emitting source array 44. In a preferred embodiment, the system 10 incorporates the DLED-660/880-CSL-2 dual optical emitter combinations from OSI Optoelectronics. This dual emitter combines a red (660 nm) and infrared (880 nm) LED into a single package. Each red/infrared LED pair requires a 20 mA current source and have a 2.4/2.0V forward voltage respectively. It is appreciated that other light sources may also be used.

In order to measure a photoplethysmograph, the light reflected from the LED array 44 is detected by the photodiode array 46. In a preferred embodiment, the PIN-8.0-CSL photodiode from OSI Optoelectronics is used. This photodiode has a spectral range of 350 nm to 1100 nm and has a responsivity of 0.33 and 0.55 to 660 nm and 900 nm light respectively.

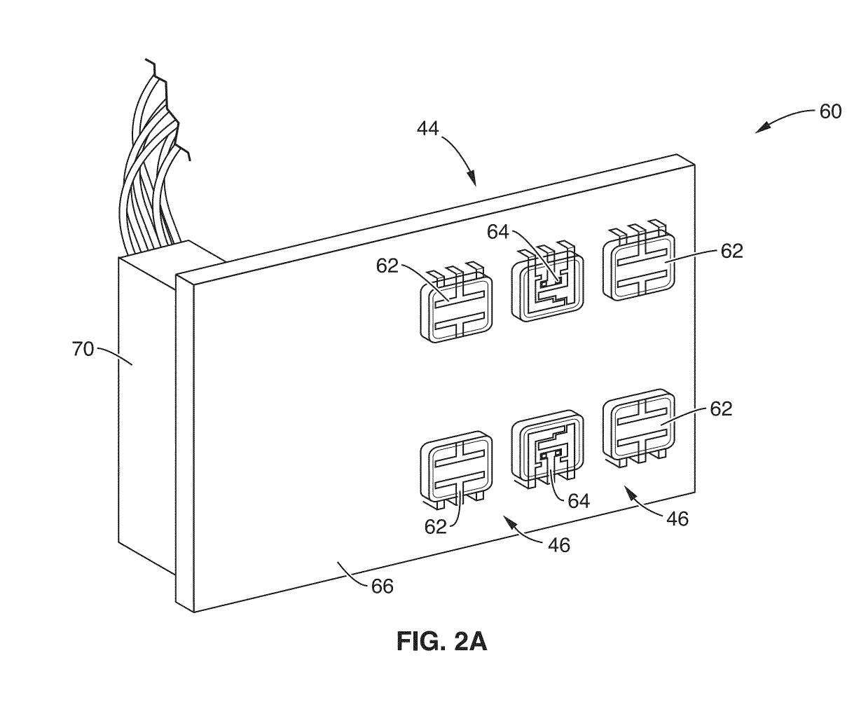

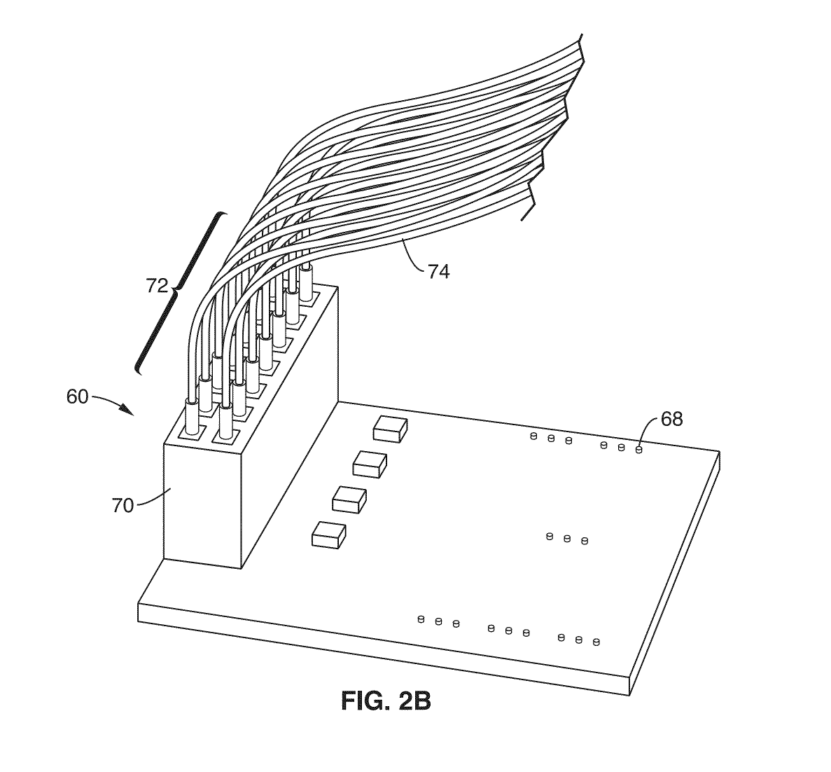

FIGS. 2A and 2B illustrate front and right perspective views of the perfusion hardware printed circuit board (PCB) 60. PCB 60 comprises LED array 44 of two LED pairs 64 spaced between two arrays 46 of photodiodes 62. The board 60 also comprises pressure sensor 50 to monitor the applied pressure on the target tissue 52.

As shown in FIG. 2A, the optical sensors (e.g. LED array 44 and photodiode array 46) are located on the front side 66 of the PCB 60 and are configured to face and press onto (either directly or adjacently with respect to transparent cover (not shown)) the target tissue 52.

Referring to FIG. 2B, driving circuitry, e.g. connector head 70, are located on the back side 68 of the PCB 60 safely out of contact with the test subject, and the front of the PCB (right) which houses the sensor portion of the array. The arrays 44, 46 are located such that connector head 70 and corresponding leads 72 and cables 74 (which couple to the data acquisition unit 40) do not interfere with using the device.

The arrays 44, 46 are shown in FIG. 2A as two LED's 64 positioned between four photodiodes 62. However, it is appreciated that the array may comprise any number of and planar configuration of at least one LED emitter 64 and one photodiode receiver.

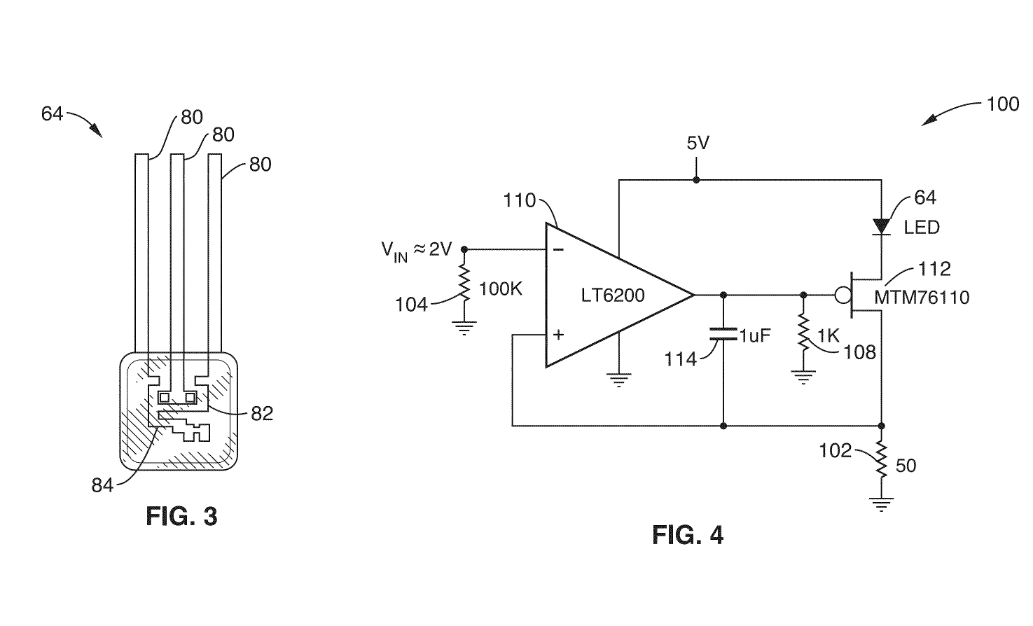

FIG. 3 illustrates an exemplary LED emitter 64 (OSI Optoelectronics DLED-660/880 CSL-2) having 660 nm red emitter 84 and 880 nm Infrared emitter 82.

FIG. 4 illustrates LED driver circuit 100 in accordance with the present invention. LED driver circuit 100 is configured to allow the red LED 88 and infrared LED 82 in the LED package 64 to be driven independently, even though the LEDs are common anode, sharing a V_{DD} connection via leads 80.

Driver circuit 100 includes a low-noise amplifier 110 coupled to the LED 64. In a preferred embodiment, the amplifier 110 comprises a LT6200 chip from Linear Technologies. However, it is appreciated that other amplifiers available in the art may also be employed. LED driver circuit 100 further comprises a p-channel MOS field-effect transistor (FET) 112 (e.g. MTM76110 by Panasonic), which provides negative feedback. As voltage is increased at the input, so is the voltage across the 50 ohm resistor 102. This results in larger current draw, which goes through the LED 64, making it brighter. At 2V, approximately 40 mA is drawn through the LED 64, providing optimal brightness. If the voltage at the input is increased too far, the voltage drop across the LED 64 will be insufficient to turn it off, but there will still be a large amount of current flowing through the LED 64 and resistor 102, resulting in large heat buildup. For this reason, the input voltage is ideally kept below 3V to minimize overheating and prevent component damage. If the input to the op-amp 110 is floated while the amp 110 is powered, a 100 k pull-down resistor 104 at the input and 1 k load resistor 108 at the output ensure that the circuit 100 remains off. The 1 k load resistor 108 also ensures that the amp 110 is able to provide rail to rail output voltage. The 1 uF capacitor 114 ensures that the output remains stable, but provides enough bandwidth for fast LED 64 switching. To provide further stabilization, the driver circuit 100 may be modified to include Miller compensation on the capacitor 114. This change improves the phase margin for the driver circuit 100 at low frequencies, allowing more reliable operation.

FIG. 5 illustrates an exemplary photodiode read circuit 120 configured for reading the signal from photodiode sensor array 46. In a preferred embodiment, the photodiode 62 may comprise an OSI Optoelectronics PIN-8.0-DPI photodiode, PIN-4.0DPI photodiode, or alternatively PIN-0.8-DPI photodiode which has lower capacitance for the same reverse bias voltage.

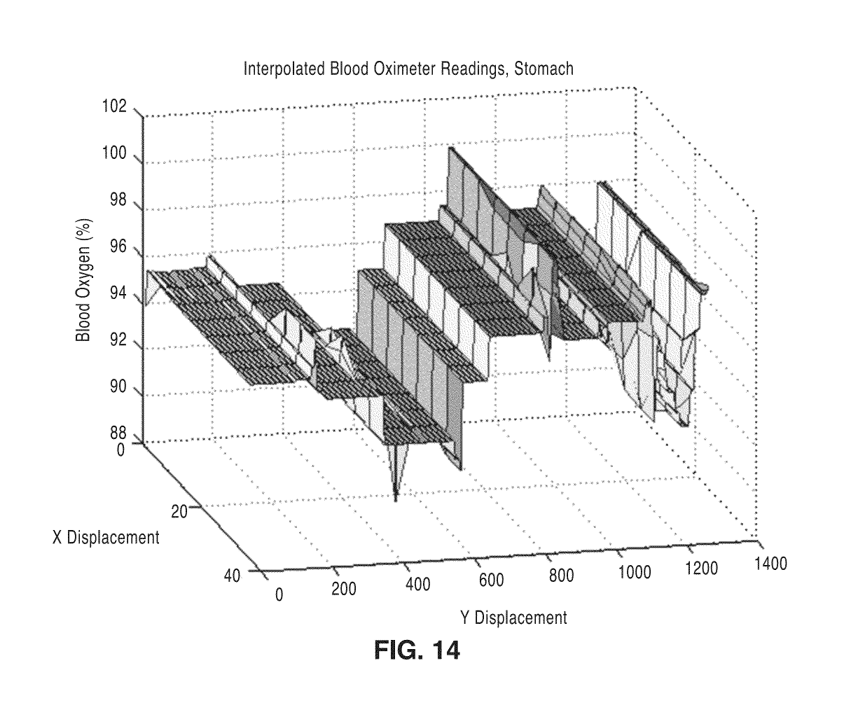

The photodiode read circuit 120 operates via a simple current to voltage op-amp 124 as shown in FIG. 14. The positive input pin of the op-amp 124 (e.g. LT6200 from Linear Technologies) is driven by a voltage divider 122, providing 2.5V (half of V_{DD}). The negative pin is hooked up to the photodiode 62, which is reverse biased, and through feedback to the output of the amplifier 124.

The feedback is controlled by a simple low pass filter 126 with a 2.7 pF capacitor 129 and a 100 kilo-ohm resistor 130. The 0.1 uF capacitor 128 is used to decouple the voltage divider from ground. The circuit amplifies the current output of the photodiode and converts it to voltage, allowing the data acquisition unit to read the voltage via its voltage input module.

It is appreciated that the individual components of the LED driver circuit 100 and photodiode read circuit 120 are shown for exemplary purposes only, and that other models, or types of components may be used as desired.

In one embodiment of the present invention, the data acquisition controller 40 comprises National Instruments CompactRIO 9014 real-time controller coupled with an NI 9104 3M gate FPGA chassis. The data acquisition controller 40 interfaces with the LED arrays 44 and photodiodes 46 using three sets of modules for current output, current input, and voltage input.

In one embodiment, the controller 40 comprises a processor, real-time operating system, memory, and supports additional storage via USB (all not shown). The controller 40 may also include an Ethernet port (not shown) for connection to the user interface PC 154. The controller 40 comprises an FPGA backplane, current output module (e.g. NI 9263), current input module (e.g. NI 9203), and voltage input module (e.g. NI 9205) allowing multiple voltage inputs from photodiode/amplifier modules.

The POM system 10 preferably employs a pressure sensor 50 to measure pressure and ensure consistent results (e.g. 1 lb. Flexiforce sensor). Due to the confounding effect varying pressure can have on plethysmograph measurements, readings from the pressure sensor 50 provide a metric from which the user can apply the sensor hardware 16 to the patient's skin 52.

The pressure sensor 50 is preferably attached behind the LED array 44, and measures the pressure used in applying it to a target location. The pressure sensor 50 is preferably configured to deliver accurate measurements of pressure in a specified range, e.g. a range from zero to approximately one pound, which encompasses the range of pressures that can reasonably be applied when using the POM sensing hardware 16.

The pressure sensor 50 is used to guide the user into operating the scanner 16 more consistently, so that the sensor/scanner 16 is positioned in a similar manner every measurement. The oximetry data that is taken is thus verified to be accurately taken by readings from the pressure sensor 50.

In a preferred embodiment, the pressure sensor 50 is calibrated in order to ensure that the pressure sensor gives repeatable, well understood measurements that can be directly translated into raw pressure values. FIG. 6 illustrates a calibration setup 140 for calibration of the pressure sensor 50. A rubber pressure applicator 144 was filed down to a flat surface, and used to distribute the weight on the pressure sensitive region of the Flexiforce sensor 50. A weight 142 was used to distribute weight over the active region of the sensor 50. An experiment was conducted using 4 weights in a range from 50 g to 500 g. Pressure was applied directly to the pressure sensor 50 via applicator 144, and its outputs recorded.

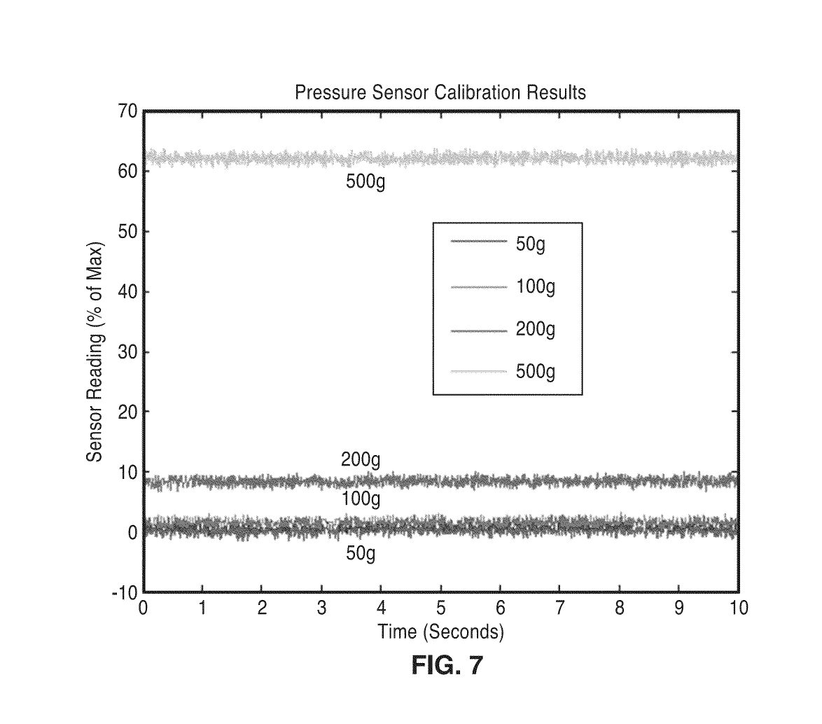

The results in FIGS. 7-10 show a nonlinear but steady trend, which data can be used to translate any future measurement from the pressure sensor into an absolute pressure value.

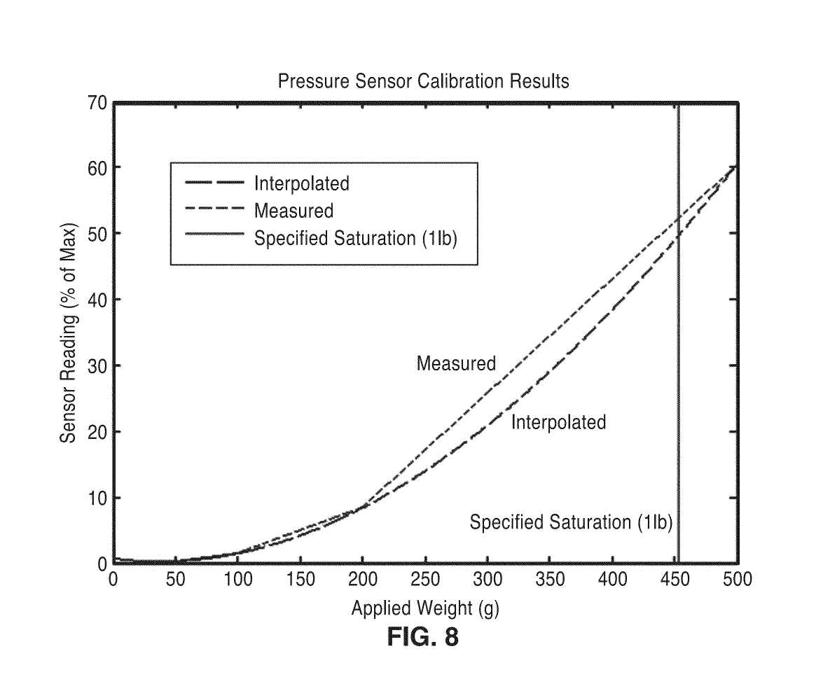

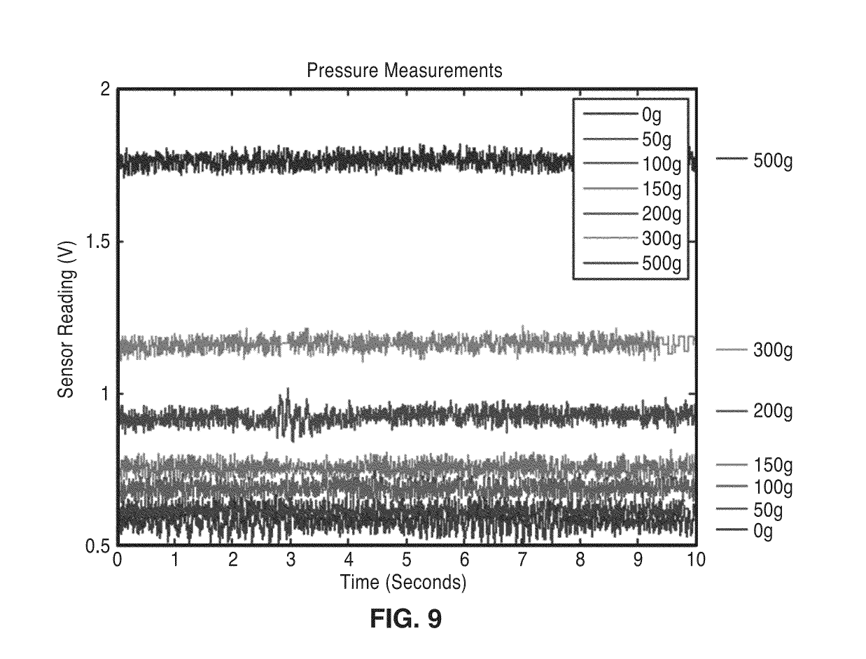

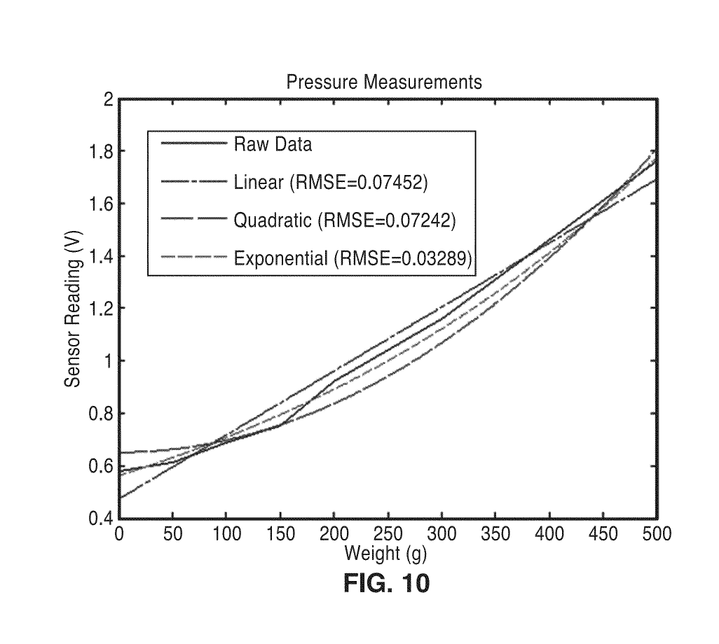

FIG. 7 shows a plot of results from the pressure verification trials of weights of 50 g, 100 g, 200 g and 500 g on a single sensor. FIG. 8 is a plot showing measured pressure response curve, interpolated curve (exponential), and the point where the pressure sensor is specified to saturate. FIG. 9 shows results from pressure verification trials on a second 1-pound sensor. For this experiment, additional intermediate weight levels (e.g. 150 g and 300 g) were applied. FIG. 10 is a plot showing raw pressure response curves, and various fits. The exponential fit serves as the best fit for both sensors tested.

While the system 10 optimally uses data from the pressure sensor 50 to verify proper disposition of the scanner on the target tissue site 52, it is appreciated that in an alternative embodiment the user may simply forego pressure monitoring and monitor pressure manually (e.g. tactile feel or simply placing the scanner 16 on the tissue site 52 under gravity).

Referring to FIG. 11, the user preferably interacts with the data acquisition and control unit 40 through a PC 154 running the processing module 12 and application module 14 comprising graphic user interface 36 (e.g. LabVIEW or the like). In a preferred embodiment, the PC 154 communicates with the data acquisition unit 40 over via an Ethernet connection (not shown). Alternatively, PC 154 communicates with the data acquisition unit 40 via a wireless connection (not shown) such as WIFI, Bluetooth, etc. Data files generated on the data acquisition unit 40 may also be transferred to the PC 154 over an FTP connection for temporary storage and further processing.

With respect to the PC 154 interface shown in FIG. 11, the individual LED's 64 of LED array 44 project light at wavelengths keyed for hemoglobin, and the photodiode sensors 62 measure the amount of light that passes through and is reflected from tissue 52. The data acquisition unit 40 generally comprises a digital TTL output 152 coupled to the LED's 64 and analog DC input 150 for photodiodes 62. The signal processing module 12 then further processes and filters this data, which is then transmitted to the graphical user interface 36 for further processing and visualization. The data may then be converted into a Plethysmograph waveform to be displayed.

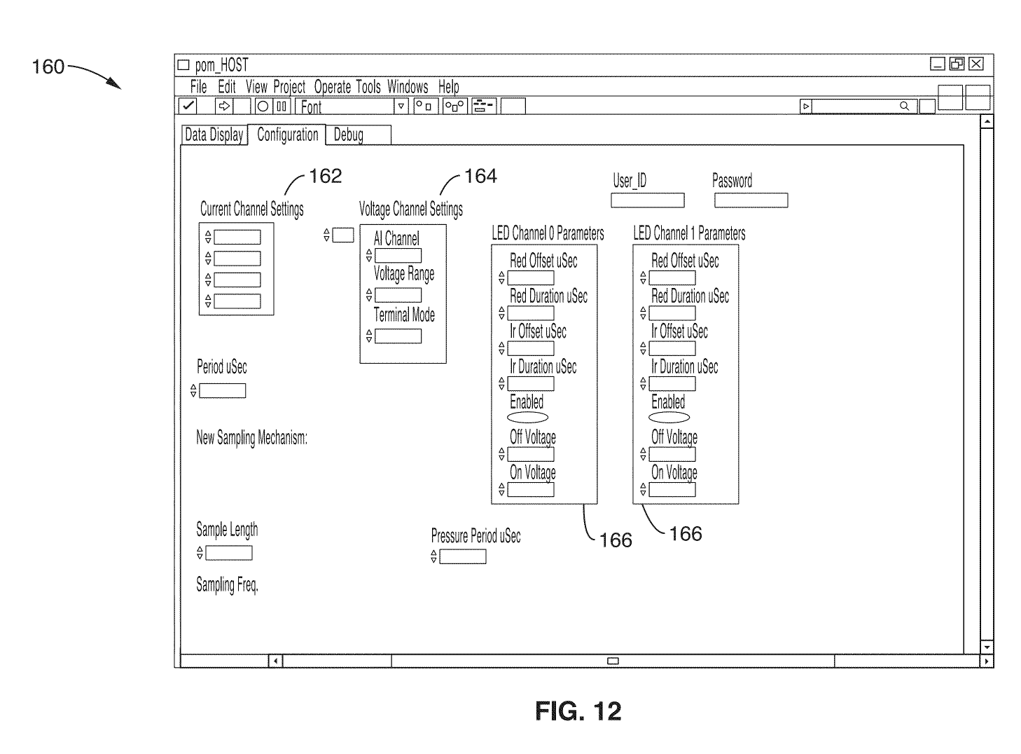

FIG. 12 shows a screenshot 160 of the hardware configuration module 34 interface. Inputs can be selected for adjusting the LED array 44 parameters in fields 166, voltage channel settings in fields 164, current channel settings in fields 162, in addition to other parameters such as the sampling period, pressure sampling period, etc.

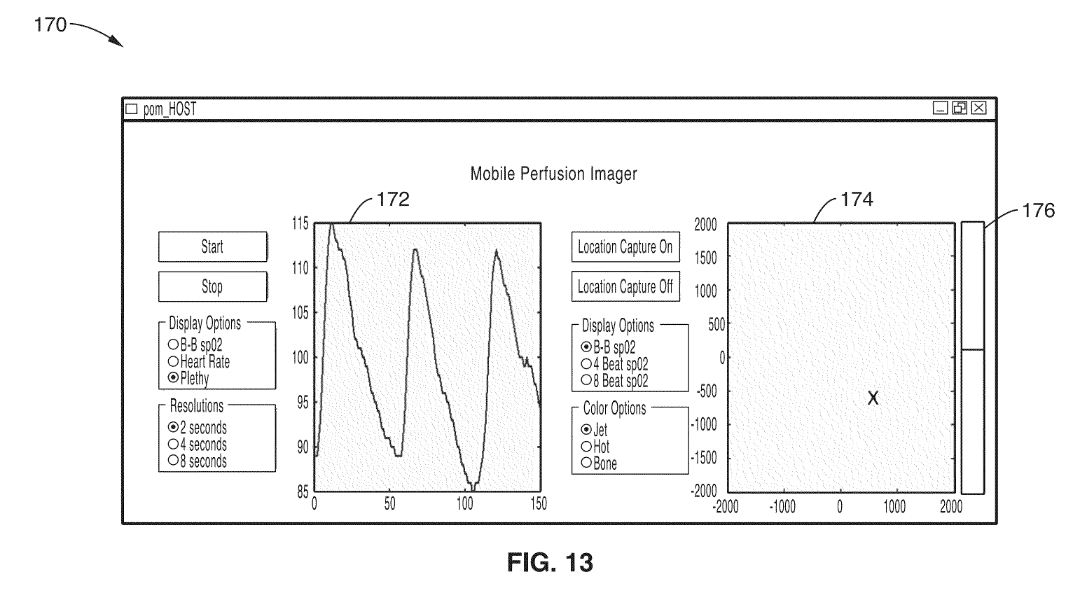

FIG. 13 shows a screenshot 170 of the graphical user interface 36 that also serves as data management and explorer to allow a user to easily read the perfusion sensors, and observe a variety of signals. The screenshot 170 shows integration of the data captured from blood oximetry sensors (photodiode array 46 and LED array 44), from pressure sensor 50, and the tracking/position data captured by the scanning the photodiode array 46 and LED array 44. The screenshot 170 shows a first window 172 that displays the Plethysmograph waveform (2 seconds shown in FIG. 13), and a second window 174 showing the absolute x and y axis movement that has been performed with the scanner. The graphical user interface 36 is also able to map this to the measured SPO₂ data (e.g. via toggling one of the display windows 172 and 174). The bar 176 on the right of the screenshot 170 is the pressure gauge from pressure sensor 50 readings, showing approximately half of maximum pressure being applied. The gauge 176 preferably displays how much pressure the user is applying versus the maximum measurable pressure in a color coded bar (as more pressure is applied the bar changes from blue to green to red). The gauge 176 is preferably mapped to optimum pressure values for different locations.

In order to provide a more informative map of perfusion in a local region, interpolation of blood oximeter data may be conducted using sensor tracking data. The optical oximeter sensor 16 provides absolute SPO₂ readings, giving the percent of blood that is oxygenated. This information, when associated with the location it was taken from, can be used to generate a map of blood oxygenation. In a preferred embodiment, the LED array 44 used for generating SPO₂ readings is also used for determining location. However, it is appreciated that another optical sensor, e.g. laser (not shown), may be used to obtain location readings independently of the LED SPO₂ readings. In such configuration, a low-power laser (similar to a laser-tracking mouse) is used to image a small area at very fast intervals, and then detects movement by how that image has shifted. This information is then converted to two dimensional ‘X’ and ‘Y’ position and displacement measurements.

In a preferred embodiment, interpolation is performed via a Kriging algorithm, and data points are mapped using the oximeter sensor 16 to track movement of the sensor 16 over the test area. Kriging is a linear least squares interpolation method often used for spatially dependent information. The interpolation is used to fill in the blank spots that a scan may have missed with estimated values. The interpolated data is compiled into a color coded image, and displayed to the user. This allows an accurate, anisotropic interpolation of the raw data, which makes the end result much easier to visualize. An example interpolation is shown in FIG. 14. Movement of the sensor hardware 16 was mostly one dimensional in this example, resulting in a linear trend across the x axis. This is due to the low variance of points in that direction (note the total displacement of approximately 40 in the X direction compared to 1400 in the Y).

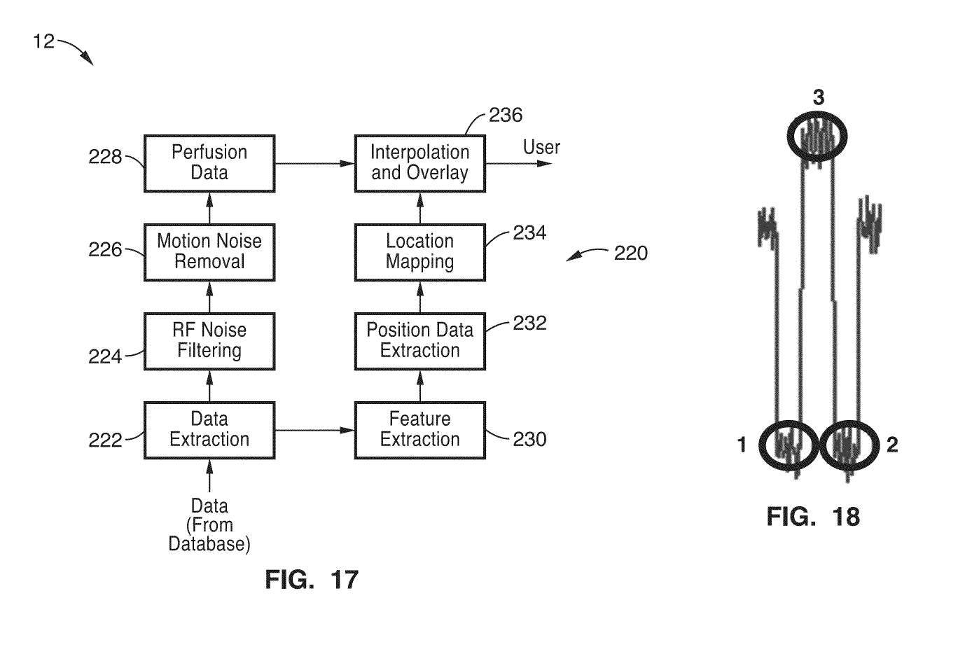

To aid in visualizing the collected blood oximetry data, the processing software 12 preferably includes a feature extraction module 28 that that can detect markers on a picture, and then properly align and overlay blood oximetry data 26 (see FIGS. 1, 17). In a preferred method, the feature extraction module 28 takes images (e.g. pictures taken from a camera of the scan site), and superimposes the perfusion data directly over where it was taken from.

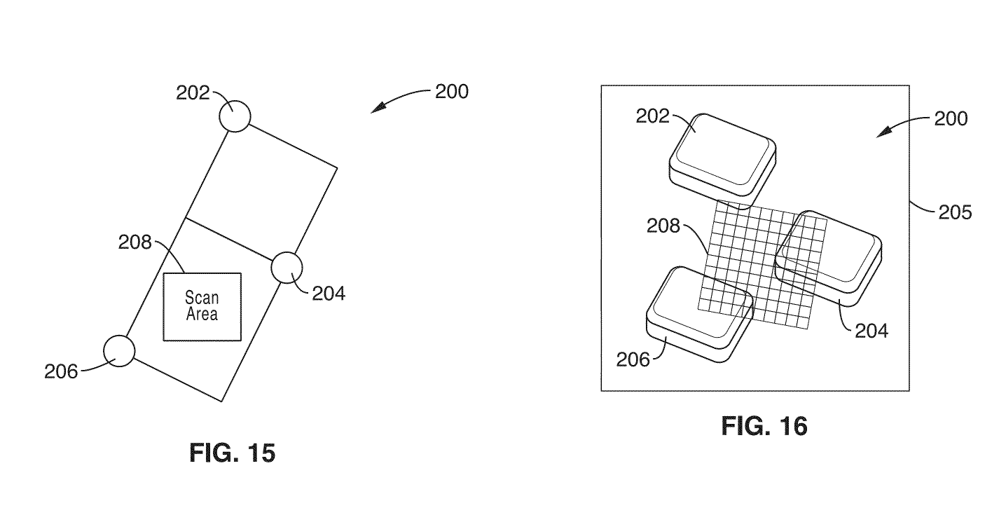

FIG. 15 shows a schematic diagram of a marker pattern 200 used for testing the feature extraction module 28. FIG. 16 illustrates the setup of FIG. 15 overlaid on an image 205. Three markers (202, 204 and 206) were used as delimiting points for a given scan area 208. A first marker 202 was used to determine rotation angle for the image. A second marker 206 was used to determine the left boundary (image position) for the image. A third marker 204 was used to determine the width of the image. The markers (202, 204 and 206) can be any color, but green is the ideal color, as it is easily distinguished from all skin tones. For a clear illustration of the feature extraction software, small plastic green boxes were used to represent points 202, 204, and 206 (see FIG. 16), and the image 205 was quickly edited to place three of them in a likely pattern. Aside from this manipulation, all other images were generated on the fly by the software. A grid 208 was used as sample data, to more clearly illustrate what is being done by the tool.

In one embodiment a mobile application (not shown) may be used to facilitate easy capture and integration of pictures for the processing software 12. The application allows a user to quickly take a picture with a mobile device (e.g. smartphone, or the like) and have it automatically sent over Bluetooth for capture by the processing software 12. The picture may then be integrated with the mapping system.

FIG. 17 illustrates a block diagram of a method 220 for outputting a mapped and interpolated perfusion image (e.g. with processing module 12). An example of code for carrying out method 220 may be found in the Source Code Appendix attached hereto. It is appreciated that the provided code is merely one example of how to perform the methods of the present invention.

Acquired data from the data acquisition unit 40 (which may be stored on server 32) is first extracted at step 222 (via processing scripts 24). This extracted data is then used for simultaneously extracting location data, perfusion data and pressure data from each measurement point. The processing software 12 may simultaneously sample location, perfusion, and pressure readings (e.g. at 3 Hz interval), in order to creating a matching set of pressure, position, and blood oxygen measurements at each interval.

In order to generate useful information and metrics from the raw data recorded by the perfusion module 228, a number of algorithms are used.

At step 230, features are extracted from the data (e.g. via the feature extraction module 28). Position data corresponding to the hardware sensor 16 location is then mapped at step 232. After a scan has been completed, the oximetry data is mapped at step 234 to appropriate coordinates corresponding to the obtained sensor position data from step 232. At step 236, the mapped data is interpolated (e.g. using the Kriging algorithm shown in FIG. 14). The interpolated data may be compiled into a color coded image, and displayed to the user, and/or the perfusion data may then overlayed on a background image (e.g. image 205) of the scan site as described in FIGS. 15 and 16.

On the perfusion side, RF noise filtering is then performed on the extracted data at step 224. Motion noise is then removed at step 226 to obtain the perfusion data at step 228. Steps 224 and 226 may be performed via filtering module 22.

In a preferred method illustrated in FIG. 18, heterodyning is used to help eliminate in-band noise. The data recorded from when the LED arrays 44 are off is subtracted from adjacent data from when LED arrays 44 are on (subtraction method). This creates high frequency noise, but removes low frequency in band noise, which is a larger issue. The additional high frequency noise that is introduced is then filtered out by a low pass filter. The algorithms are configurable to allow the preservation of high frequency information of the PPG signals.

As illustrated in FIG. 18, relevant noise information from the areas marked 1 and 2 is used to calculate the noise that appears in area 3. This may be done by either the single-sided method or the doubled-sided method.

For the single sided method, only the preceding noise information from area 1 is used, and the relevant noise level is assumed to be the same in area 1 and 3. For the double sided method, noise from areas 1 and 2 is averaged. Finally, interpolation of the noise at 3 is attempted via interpolation, using the data from all available noise periods, preceding and following the target data point (3). The measurement data is averaged in these areas to generate a single point for each LED 64 pulse. The result is then low-pass filtered at the end to remove high frequency noise.

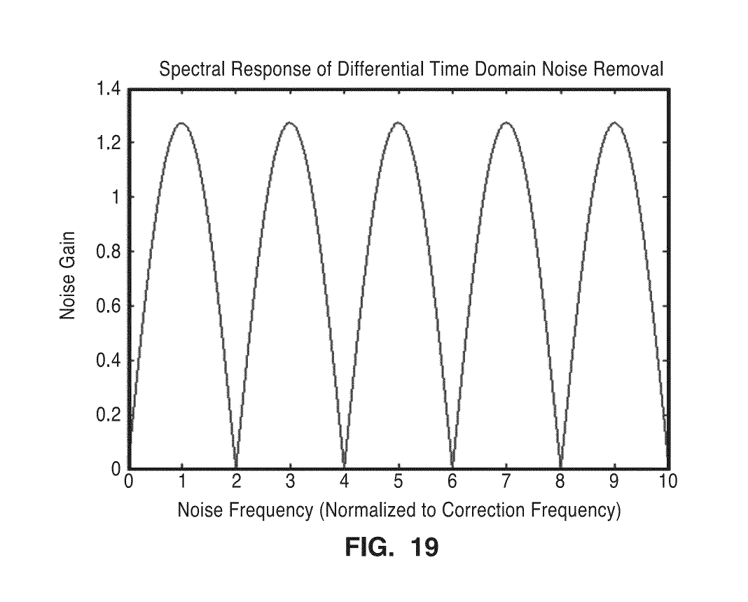

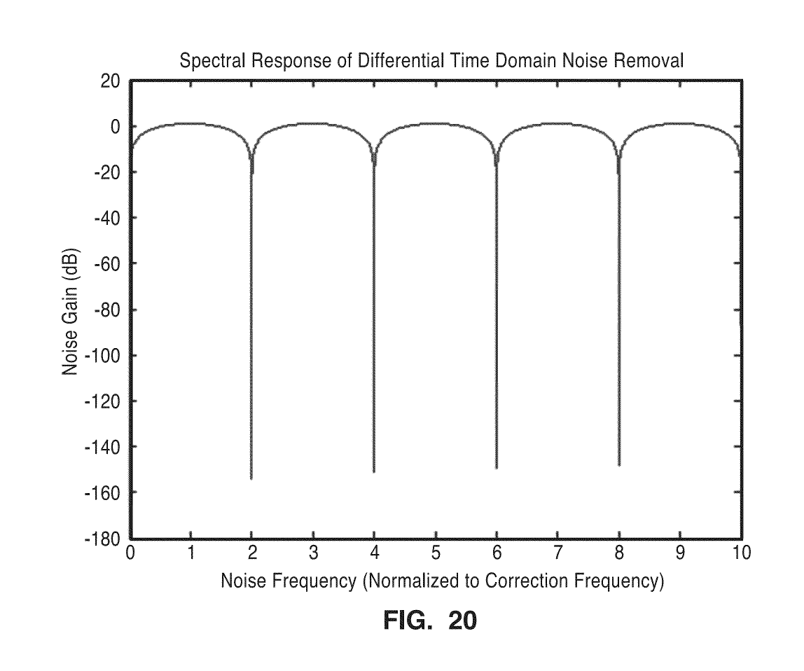

FIG. 19 is a plot of the theoretical response of the subtraction method of FIG. 18 in relation to noise and correction frequency, determined by adding sinusoidal noise of a wide range of frequencies to a square wave signal, applying the noise cancellation method (correction method), and measuring the ratio of remaining noise to original noise. Measurements were averaged across all phases for a given frequency. FIG. 20 is a plot of the frequency response of the subtraction method shown on a dB scale.

For the frequency response plots shown in FIGS. 19 and 20, the frequency is normalized to the frequency of the simulated LED drive signal, with 1 meaning the noise is the same frequency as the drive signal and 2 meaning it is double the drive frequency, and so forth.

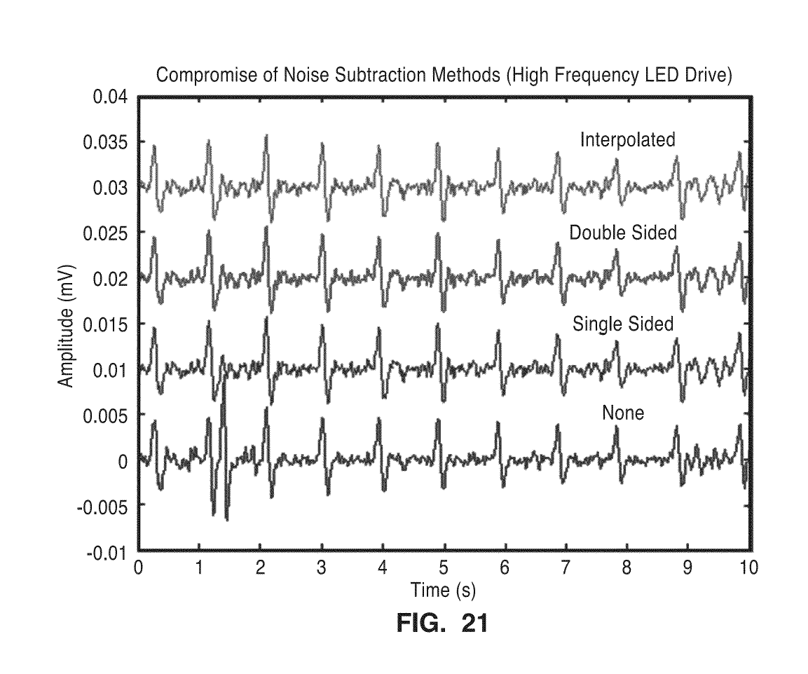

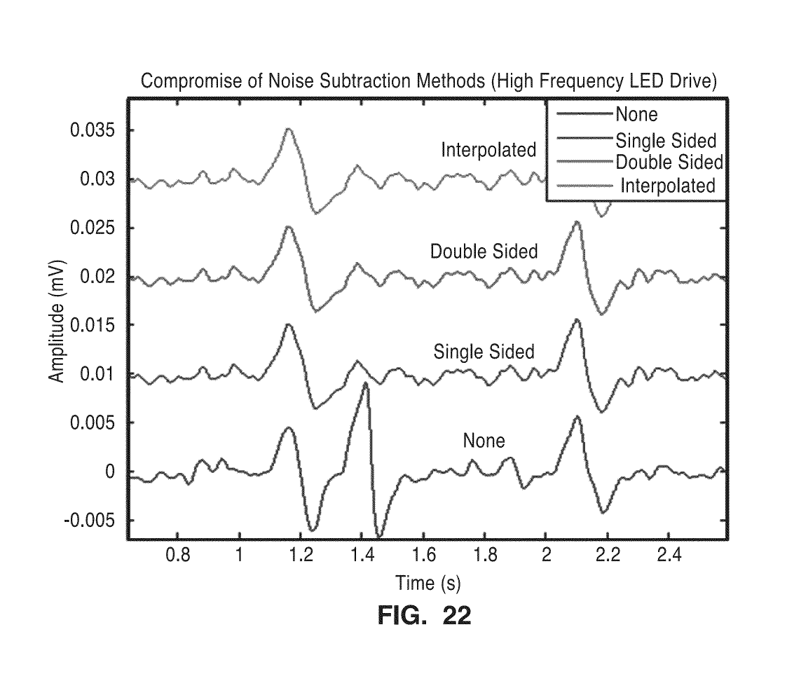

FIGS. 21 and 22 are plots showing the extracted plethysmograph signals employing the aforementioned noise cancelation (subtraction) method of FIG. 18 on a high frequency LED drive signal compared to the scenario when no noise cancellation technique is performed. FIG. 21 shows results from employing noise subtraction on a high frequency LED drive signal, and averaging several LED drive periods to obtain similar data rates as before. Note the successful noise reduction at around 1.5 s. FIG. 22 is a zoomed version of FIG. 21, showing the noise spike that is removed by differential noise subtraction. These plots show that the noise subtraction method of the present invention is effective in removing in band noise.

Frequency domain analysis/experiments were performed with the frequency domain signals of the plethysmograph measurements. The experiments revealed not only high magnitude elements at the heart rate frequency, but also its harmonics. This appears fairly consistent between locations.

In order to verify that the harmonics shown in the frequency domain were not the result of noise or jitter, but represented real components of the pulse waveform, a sinusoid wave was constructed. The sinusoid was created by summing sinusoids at the frequency for each separate pulse waveform peak. This superposition was intended to model the effects of frequency jitter in the waveform, while removing any frequency components due to the pulse waveform shape.

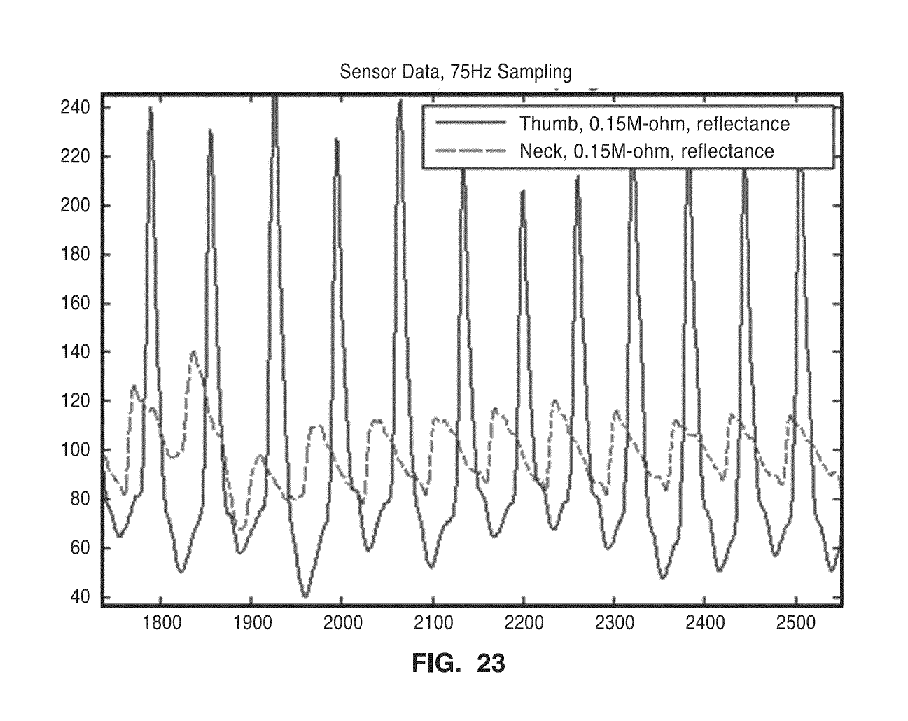

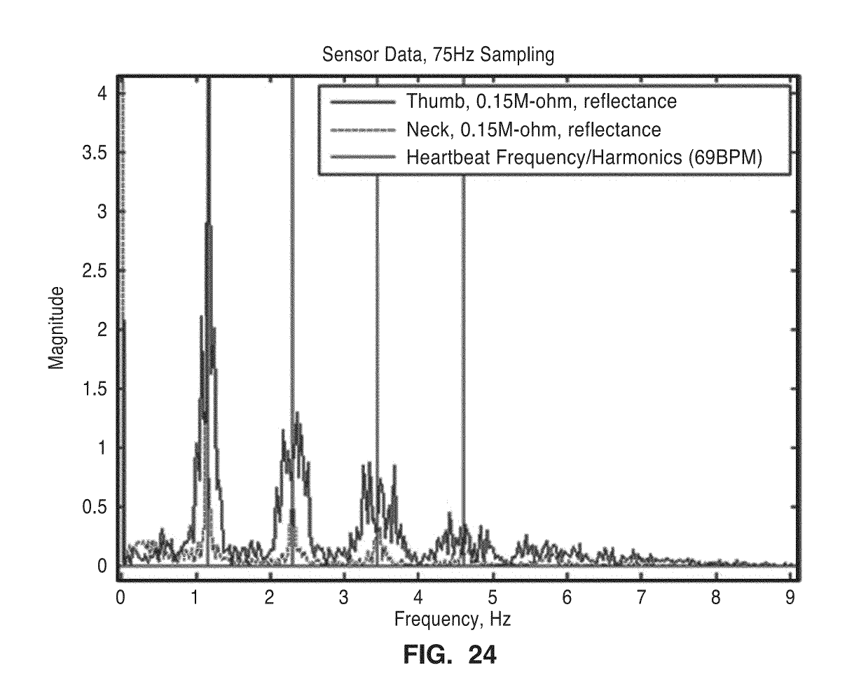

A comparison of signals is shown in FIGS. 23 and 24. FIG. 23 shows a sample of the time domain signals used for comparison. Neck measurements were compared to thumb measurements, taken at equal pressure. FIG. 24 shows the frequency domain representation of the measured signals. Note the second harmonic at 128 BPM (2.13 Hz), the third harmonic at 207 BPM (3.45 Hz), etc. The results demonstrate that the harmonics shown below are indeed intrinsic to the pulse waveform, and are not the result of noise or frequency jitter.

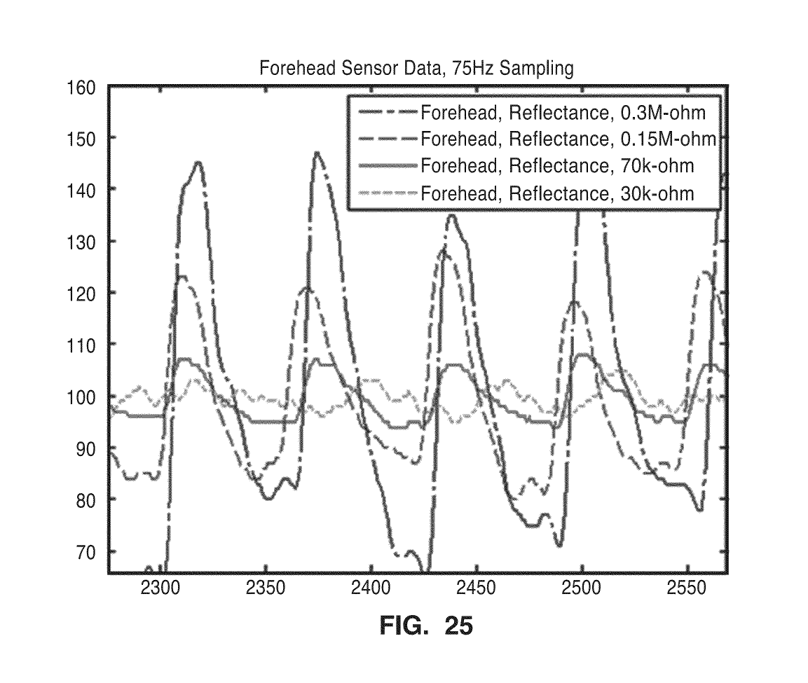

Experiments were performed on number of body locations, including neck, thumb and forehead using the perfusion system 10 of the present invention. Samples of extracted plethysmograph signals are reported in FIGS. 25-27, which clearly show that perfusion system successfully removes the motion and ambient noises and extracts the plethysmograph signal from different body location.

FIG. 25 shows results from extracted plethysmograph signals of the forehead. Pressure values are given in terms of resistance measured using the pressure sensor. Smaller resistances indicate higher applied pressures.

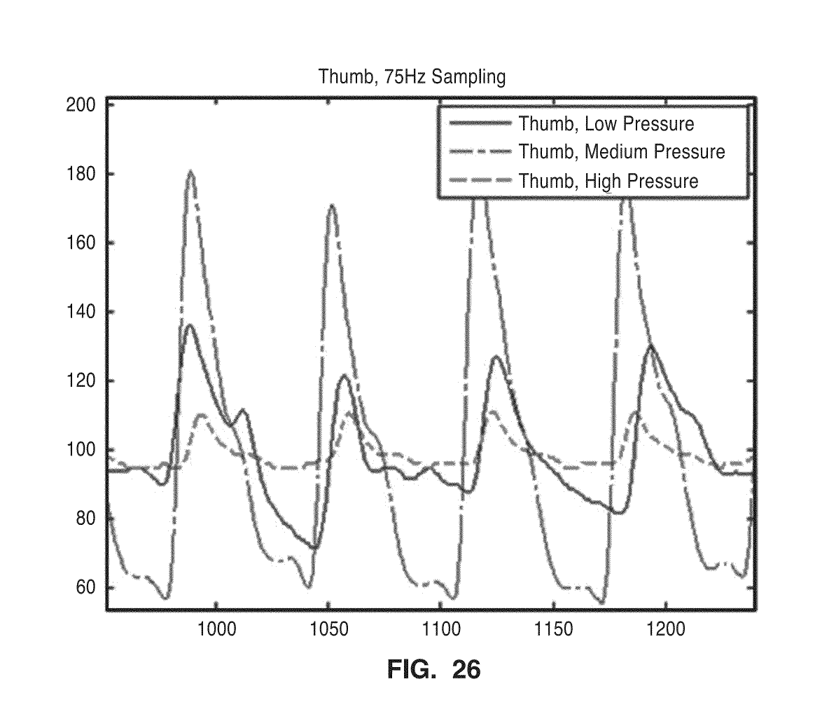

FIG. 26 shows a comparison of readings of extracted plethysmograph signals from under the knuckle on the thumb. All factors except pressure were held constant between measurements. A moderate pressure clearly results in a better waveform.

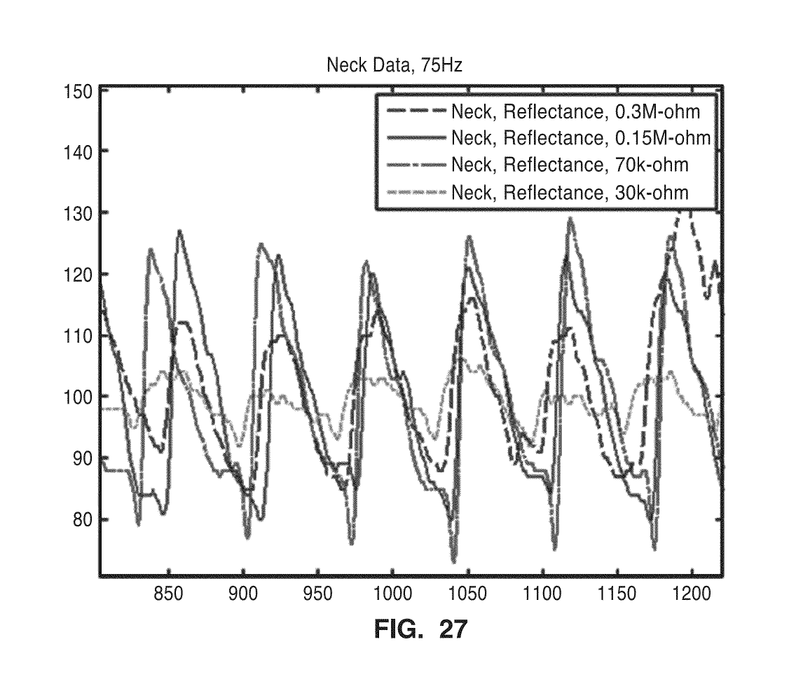

FIG. 27 shows results from varying pressure using the reflectance sensor on the neck. The following experiments show the importance of the integration and fusion of applied pressure with perfusion signal in this system, since the pressure with which the sensor array is applied to the target tissue has a major impact on the perfusion readings as shown in the following figures. It appears that the neck and thumb give best results when moderate (0.15 M to 70 k-ohm) pressure is applied, while the forehead yield best results with low pressure (above 0.15 M-ohm). This may be a result of the neck and thumb being softer tissue than the forehead.

The perfusion system 10 was also tested on a black tape, as a means to mark locations on tissue. Black tape was used to test as a marker on the skin. The sensor was used to measure signals on the tape, and just to the side of it. An impression on the skin can be seen where the reflectance sensor was used off the tape.

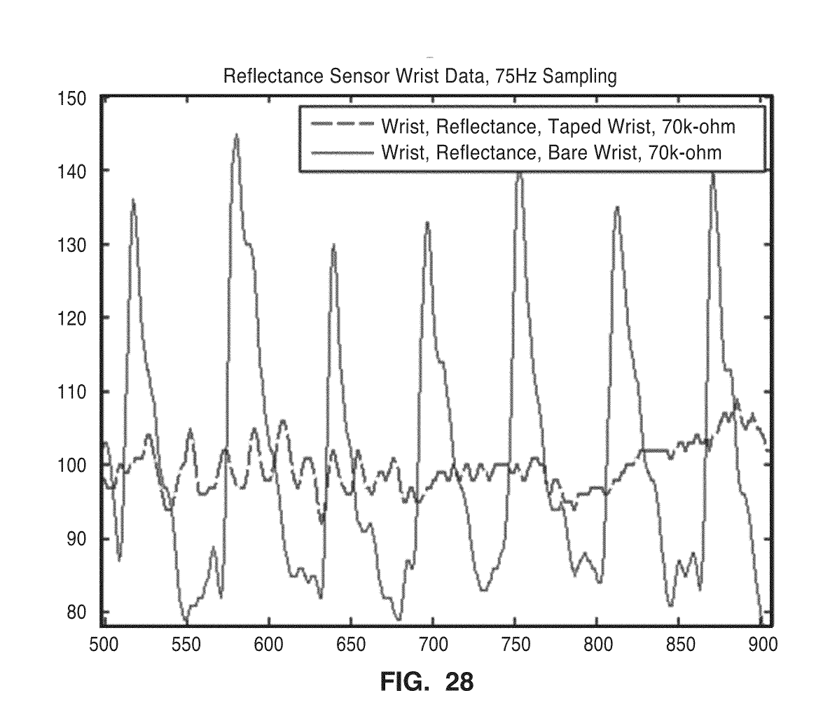

FIG. 28 shows the results from both over and to the side of the black tape. The results show that using a simple piece of black tape is effective in causing large signal differences, and could therefore be used as a marker for specific body locations.

Embodiments of the present invention may be described with reference to flowchart illustrations of methods and systems according to embodiments of the invention, and/or algorithms, formulae, or other computational depictions, which may also be implemented as computer program products. In this regard, each block or step of a flowchart, and combinations of blocks (and/or steps) in a flowchart, algorithm, formula, or computational depiction can be implemented by various means, such as hardware, firmware, and/or software including one or more computer program instructions embodied in computer-readable program code logic. As will be appreciated, any such computer program instructions may be loaded onto a computer, including without limitation a general purpose computer or special purpose computer, or other programmable processing apparatus to produce a machine, such that the computer program instructions which execute on the computer or other programmable processing apparatus create means for implementing the functions specified in the block(s) of the flowchart(s).

Accordingly, blocks of the flowcharts, algorithms, formulae, or computational depictions support combinations of means for performing the specified functions, combinations of steps for performing the specified functions, and computer program instructions, such as embodied in computer-readable program code logic means, for performing the specified functions. It will also be understood that each block of the flowchart illustrations, algorithms, formulae, or computational depictions and combinations thereof described herein, can be implemented by special purpose hardware-based computer systems which perform the specified functions or steps, or combinations of special purpose hardware and computer-readable program code logic means.

Furthermore, these computer program instructions, such as embodied in computer-readable program code logic, may also be stored in a computer-readable memory that can direct a computer or other programmable processing apparatus to function in a particular manner, such that the instructions stored in the computer-readable memory produce an article of manufacture including instruction means which implement the function specified in the block(s) of the flowchart(s). The computer program instructions may also be loaded onto a computer or other programmable processing apparatus to cause a series of operational steps to be performed on the computer or other programmable processing apparatus to produce a computer-implemented process such that the instructions which execute on the computer or other programmable processing apparatus provide steps for implementing the functions specified in the block(s) of the flowchart(s), algorithm(s), formula(e), or computational depiction(s).

From the discussion above it will be appreciated that the invention can be embodied in various ways, including the following:
1. An apparatus for monitoring perfusion oxygenation of a target tissue region of a patient, comprising: a scanner comprising: a planar sensor array; the sensor array configured to be positioned in contact with a surface of the target tissue region; the sensor array comprising one or more LED's configured to emit light into the target tissue region at a wavelength keyed for hemoglobin; the sensor array comprising one or more photodiodes configured to detect light reflected from the LED's; and a data acquisition controller coupled to the one or more LED's and to the one or more photodiodes for controlling the emission and reception of light from the sensor array to obtain perfusion oxygenation data associated with the target tissue region.
2. The apparatus of embodiment 1, the scanner further comprising: a pressure sensor coupled to the sensor array; the pressure sensor configured to obtain pressure readings of the sensor array's contact with a surface of the target tissue region; wherein the scanner is configured to obtain pressure sensor readings while obtaining perfusion oxygenation data to ensure proper contact of the scanner with the surface of the target tissue region.
3. The apparatus of embodiment 2: wherein the pressure sensors and sensor array are connected to a first side of a printed circuit board (PCB); and wherein the data acquisition controller is connected to the PCB on a second side opposite said first side.
4. The apparatus of embodiment 1, wherein each LED comprises dual emitters configured for emitting red (660 nm) and infrared (880 nm) light.
5. The apparatus of embodiment 4: wherein the one or more of the LED's are coupled driver circuit; and wherein the driver circuit is configured to allow the red LED emitter and infrared LED emitter to be driven independently while sharing a common anode.
6. The apparatus of embodiment 5, wherein the driver circuit comprises an amplifier; and a field-effect transistor configured for providing negative feedback.
7. The apparatus of embodiment 2, further comprising: a processing module coupled to the data acquisition controller; the processing module configured to control sampling of the pressure sensor and sensor array for simultaneous acquisition of pressure sensor data and perfusion oxygenation data.
8. The apparatus of embodiment 7, wherein the processing module is configured to obtain readings from the sensor array to obtain position data of the scanner.
9. The apparatus of embodiment 8, wherein the processing module is configured to generate a perfusion oxygenation map of the target tissue.
10. The apparatus of embodiment 8, wherein the processing module is configured to control sampling of the pressure sensor and sensor array for simultaneous acquisition of two or more data parameters selected from the group consisting of pressure sensor data, perfusion oxygenation data, and position data, to simultaneously display said two or more data parameters.
11. A system for monitoring perfusion oxygenation of a target tissue region of a patient, comprising: (a) a scanner comprising: a planar sensor array; the sensor array configured to be positioned in contact with a surface of the target tissue region; the sensor array comprising one or more light sources configured to emit light into the target tissue region at a wavelength keyed for hemoglobin; the sensor array comprising one or more sensors configured to detect light reflected from the light sources; a pressure sensor coupled to the sensor array; the pressure sensor configured to obtain pressure readings of the sensor array's contact with a surface of the target tissue region; and (b) a data acquisition controller coupled to the one or more sensors and for controlling the emission and reception of light from the sensor array to obtain perfusion oxygenation data associated with the target tissue; and (c) a processing module coupled to the data acquisition controller; (d) the processing module configured to control sampling of the pressure sensor and sensor array for simultaneous acquisition of perfusion oxygenation data and pressure sensor data to ensure proper contact of the scanner with the surface of the target tissue region.
12. The system of embodiment 11: wherein the sensor array comprises one or more LED's configured to emit light into the target tissue region at a wavelength keyed for hemoglobin; and wherein the sensor array comprises one or more photodiodes configured to detect light reflected from the LED's.
13. The system of embodiment 12: wherein each of the one or more LED's comprises dual emitters configured for emitting red (660 nm) and infrared (880 nm) light; wherein the one or more LED's are coupled to the driver circuit; and wherein the driver circuit is configured to allow the red LED emitter and the infrared LED emitter to be driven independently while sharing a common anode.
14. The system of embodiment 11, further comprising: a graphical user interface; wherein the graphical user interface is configured to display the perfusion oxygenation data and pressure sensor data.
15. The system of embodiment 14, the processing module is further configured to obtain readings from the sensor array to obtain position data of the scanner.
16. The system of embodiment 15, wherein the processing module is further configured to interpolate the position data to generate a perfusion oxygenation map of the target tissue.
17. The system of embodiment 16, wherein the processing module is configured to control sampling of the pressure sensor and sensor array for simultaneous acquisition of two or more data parameters selected from the group consisting of pressure sensor data, perfusion oxygenation data, and position data, to simultaneously display the two or more data parameters.
18. The system of embodiment 16, wherein the processing module is configured to receive an image of the target tissue, and overlay the perfusion oxygenation map over the image.
19. The system of embodiment 14, wherein the graphical user interface is configured to allow user input to manipulate settings of the sensor array and pressure sensor.
20. The system of embodiment 11, wherein the processing module further comprises: a filtering module; the filtering module configure to filter in-band noise by subtracting data recorded when the one or more light sources are in an “off” state from data recorded when the one or more light sources are in an “on” state.
21. A method for performing real-time monitoring of perfusion oxygenation of a target tissue region of a patient, comprising: positioning a sensor array in contact with a surface of the target tissue region; emitting light from lights sources in the sensor array into the target tissue region at a wavelength keyed for hemoglobin; receiving light reflected from the light sources; obtaining pressure data associated with the sensor array's contact with a surface of the target tissue region; obtaining perfusion oxygenation data associated with the target tissue region; and sampling the perfusion oxygenation data and pressure data to ensure proper contact of the sensor array with the surface of the target tissue region.
22. A method as recited in embodiment 21: wherein the sensor array comprises one or more LED's configured to emit light into the target tissue region at a wavelength keyed for hemoglobin; and wherein the sensor array comprises one or more photodiodes configured to detect light reflected from the LED's.
23. A method as recited in embodiment 22: wherein each of the one or more LED's comprises dual emitters configured for emitting red (660 nm) and infrared (880 nm) light; the method further comprising independently driving the red LED emitter and infrared LED emitter while the red LED emitter and infrared LED emitter share a common anode.
24. A method as recited in embodiment 21, further comprising: simultaneously displaying the perfusion oxygenation data and pressure sensor data.
25. A method as recited in embodiment 21, further comprising: acquiring readings from the sensor array to obtain position data of the scanner.
26. A method as recited in embodiment 25, further comprising: interpolating the position data to generate a perfusion oxygenation map of the target tissue.
27. A method as recited in embodiment 26, wherein interpolating the position data comprises applying a Kriging algorithm to the acquired position data.
28. A method as recited in embodiment 26, further comprising: sampling of the pressure sensor and sensor array for simultaneous acquisition of pressure sensor data, perfusion oxygenation data, and position data; and simultaneously displaying the pressure sensor data, perfusion oxygenation data, and position data.
29. A method as recited in embodiment 26, further comprising: receiving an image of the target tissue; and overlaying the perfusion oxygenation map over the image.
30. A method as recited in embodiment 21, further comprising: providing a graphical user interface to allow user input; and manipulating sampling settings of the sensor array and pressure sensor according to said user input.
31. A method as recited in embodiment 21, further comprising: cycling the one or more light sources between a period when the one or more light sources are on, and a period when the one or more light sources are in an “off” state; and filtering in-band noise by subtracting data recorded from when the one or more light sources are off from data from when the one or more light sources are in an “on” state.

Although the description above contains many details, these should not be construed as limiting the scope of the invention but as merely providing illustrations of some of the presently preferred embodiments of this invention. Therefore, it will be appreciated that the scope of the present invention fully encompasses other embodiments which may become obvious to those skilled in the art, and that the scope of the present invention is accordingly to be limited by nothing other than the appended claims, in which reference to an element in the singular is not intended to mean “one and only one” unless explicitly so stated, but rather “one or more.” All structural, chemical, and functional equivalents to the elements of the above-described preferred embodiment that are known to those of ordinary skill in the art are expressly incorporated herein by reference and are intended to be encompassed by the present claims. Moreover, it is not necessary for a device or method to address each and every problem sought to be solved by the present invention, for it to be encompassed by the present claims. Furthermore, no element, component, or method step in the present disclosure is intended to be dedicated to the public regardless of whether the element, component, or method step is explicitly recited in the claims. No claim element herein is to be construed under the provisions of 35 U.S.C. 112, sixth paragraph, unless the element is expressly recited using the phrase “means for.”

### SOURCE CODE APPENDIX

Appendix A contains source code that is submitted by way of example, and not of limitation, as an embodiment of signal processing in the present invention. Those skilled in the art will readily appreciate that signal processing can be performed in various other ways, which would be readily understood from the description herein, and that the signal processing methods are not limited to those illustrated in Appendix A.

What is claimed is:

1. An apparatus for monitoring perfusion oxygenation of a target tissue region of a patient, comprising:
a scanner comprising:
a planar sensor array;
the sensor array configured to be positioned in contact with a surface of the target tissue region;
the sensor array comprising one or more LED's configured to emit light into the target tissue region at a wavelength keyed for hemoglobin;
the sensor array comprising one or more photodiodes configured to detect light reflected from the LED's; and
a data acquisition controller coupled to the one or more LED's and to the one or more photodiodes for controlling the emission and reception of light from the sensor array to obtain perfusion oxygenation data associated with the target tissue region.

2. An apparatus as recited in claim 1, the scanner further comprising:
a pressure sensor coupled to the sensor array;
the pressure sensor configured to obtain pressure readings of the sensor array's contact with a surface of the target tissue region;
wherein the scanner is configured to obtain pressure sensor readings while obtaining perfusion oxygenation data to ensure proper contact of the scanner with the surface of the target tissue region.

3. An apparatus as recited in claim 2:
wherein the pressure sensors and sensor array are connected to a first side of a printed circuit board (PCB); and
wherein the data acquisition controller is connected to the PCB on a second side opposite said first side.

4. An apparatus as recited in claim 1, wherein each LED comprises dual emitters configured for emitting red (660 nm) and infrared (880 nm) light.

5. An apparatus as recited in claim 4:
wherein the one or more of the LED's are coupled driver circuit; and
wherein the driver circuit is configured to allow the red LED emitter and infrared LED emitter to be driven independently while sharing a common anode.

6. An apparatus as recited in claim 5, wherein the driver circuit comprises
an amplifier; and
a field-effect transistor configured for providing negative feedback.

7. An apparatus as recited in claim 2, further comprising:
a processing module coupled to the data acquisition controller;
the processing module configured to control sampling of the pressure sensor and sensor array for simultaneous acquisition of pressure sensor data and perfusion oxygenation data.

8. An apparatus as recited in claim 7, wherein the processing module is configured to obtain readings from the sensor array to obtain position data of the scanner.

9. An apparatus as recited in claim 8, wherein the processing module is configured to generate a perfusion oxygenation map of the target tissue.

10. An apparatus as recited in claim 8, wherein the processing module is configured to control sampling of the pressure sensor and sensor array for simultaneous acquisition of two or more data parameters selected from the group consisting of pressure sensor data, perfusion oxygenation data, and position data, to simultaneously display said two or more data parameters.

11. A system for monitoring perfusion oxygenation of a target tissue region of a patient, comprising:
(a) a scanner comprising:
a planar sensor array;
the sensor array configured to be positioned in contact with a surface of the target tissue region;
the sensor array comprising one or more light sources configured to emit light into the target tissue region at a wavelength keyed for hemoglobin;
the sensor array comprising one or more sensors configured to detect light reflected from the light sources;
a pressure sensor coupled to the sensor array;
the pressure sensor configured to obtain pressure readings of the sensor array's contact with a surface of the target tissue region; and
(b) a data acquisition controller coupled to the one or more sensors and for controlling the emission and reception of light from the sensor array to obtain perfusion oxygenation data associated with the target tissue; and
(c) a processing module coupled to the data acquisition controller;
(d) the processing module configured to control sampling of the pressure sensor and sensor array for simultaneous acquisition of perfusion oxygenation data and pressure sensor data to ensure proper contact of the scanner with the surface of the target tissue region.

12. A system as recited in claim 11:
wherein the sensor array comprises one or more LED's configured to emit light into the target tissue region at a wavelength keyed for hemoglobin; and
wherein the sensor array comprises one or more photodiodes configured to detect light reflected from the LED's.

13. A system as recited in claim 12:
wherein each of the one or more LED's comprises dual emitters configured for emitting red (660 nm) and infrared (880 nm) light;
wherein the one or more LED's are coupled to the driver circuit; and
wherein the driver circuit is configured to allow the red LED emitter and the infrared LED emitter to be driven independently while sharing a common anode

14. A system as recited in claim 11, further comprising:
a graphical user interface;
wherein the graphical user interface is configured to display the perfusion oxygenation data and pressure sensor data.

15. A system as recited in claim 14, the processing module is further configured to obtain readings from the sensor array to obtain position data of the scanner.

16. A system as recited in claim 15, wherein the processing module is further configured to interpolate the position data to generate a perfusion oxygenation map of the target tissue.

17. A system as recited in claim 16, wherein the processing module is configured to control sampling of the pressure sensor and sensor array for simultaneous acquisition of two or more data parameters selected from the group consisting of pressure sensor data, perfusion oxygenation data, and position data, to simultaneously display the two or more data parameters.

18. A system as recited in claim 16, wherein the processing module is configured to receive an image of the target tissue, and overlay the perfusion oxygenation map over the image.

19. A system as recited in claim 14, wherein the graphical user interface is configured to allow user input to manipulate settings of the sensor array and pressure sensor.

20. A system as recited in claim 11, wherein the processing module further comprises:
a filtering module;
the filtering module configure to filter in-band noise by subtracting data recorded when the one or more light sources are in an “off” state from data recorded when the one or more light sources are in an “on” state.

21. A method for performing real-time monitoring of perfusion oxygenation of a target tissue region of a patient, comprising:
positioning a sensor array in contact with a surface of the target tissue region;
emitting light from lights sources in the sensor array into the target tissue region at a wavelength keyed for hemoglobin;
receiving light reflected from the light sources;
obtaining pressure data associated with the sensor array's contact with a surface of the target tissue region;
obtaining perfusion oxygenation data associated with the target tissue region; and
sampling the perfusion oxygenation data and pressure data to ensure proper contact of the sensor array with the surface of the target tissue region.

22. A method as recited in claim 21:
wherein the sensor array comprises one or more LED's configured to emit light into the target tissue region at a wavelength keyed for hemoglobin; and
wherein the sensor array comprises one or more photodiodes configured to detect light reflected from the LED's.

23. A method as recited in claim 22:
wherein each of the one or more LED's comprises dual emitters configured for emitting red (660 nm) and infrared (880 nm) light;
the method further comprising independently driving the red LED emitter and infrared LED emitter while the red LED emitter and infrared LED emitter share a common anode

24. A method as recited in claim 21, further comprising:
simultaneously displaying the perfusion oxygenation data and pressure sensor data.

25. A method as recited in claim 21, further comprising:
acquiring readings from the sensor array to obtain position data of the scanner.

26. A method as recited in claim 25, further comprising:
interpolating the position data to generate a perfusion oxygenation map of the target tissue.

27. A method as recited in claim 26, wherein interpolating the position data comprises applying a Kriging algorithm to the acquired position data.

28. A method as recited in claim 26, further comprising:
sampling of the pressure sensor and sensor array for simultaneous acquisition of pressure sensor data, perfusion oxygenation data, and position data; and
simultaneously displaying the pressure sensor data, perfusion oxygenation data, and position data.

29. A method as recited in claim 26, further comprising:
receiving an image of the target tissue; and
overlaying the perfusion oxygenation map over the image.

30. A method as recited in claim 21, further comprising:
providing a graphical user interface to allow user input; and
manipulating sampling settings of the sensor array and pressure sensor according to said user input.

31. A method as recited in claim 21, further comprising:
cycling the one or more light sources between a period when the one or more light sources are on, and a period when the one or more light sources are off; and
filtering in-band noise by subtracting data recorded from when the one or more light sources are in an “off” state from data from when the one or more light sources are in an “on” state.
